(19) United States
(12) Patent Application Publication
(10) Pub. No.: US 2022/0079888 A1
(43) Pub. Date: Mar. 17, 2022

(54) PRE-NATAL BETA-CRYPTOXANTHIN BENEFITS CHILDREN

(71) Applicants: AGENCY FOR SCIENCE, TECHNOLOGY AND RESEARCH (A*STAR), Singapore (SG); NATIONAL UNIVERSITY OF SINGAPORE, Singapore (SG)

(72) Inventors: Mary FF CHONG, Singapore (SG); Choon Nam ONG, Singapore (SG); Jun Shi LAI, Singapore (SG); Anne Elise RIFKIN-GRABOI, Singapore (GB); Birit BROEKMAN, Amsterdam (NL)

(21) Appl. No.: 17/425,505

(22) PCT Filed: Jan. 23, 2020
(86) PCT No.: PCT/SG2020/050036

§ 371 Date: Jul. 23, 2021

(30) Foreign Application Priority Data
Jan. 23, 2019 (SG) 10201900604T

(51) Int. Cl. A61K 31/045; A61K 31/01; A61P 25/28

(52) U.S. Cl. CPC A61K 31/045; A61P 25/28; A61K 31/01

(57) ABSTRACT

Beta-cryptoxanthin administered to a pregnant woman has numerous benefits. It can decrease anxiety, and lessen the risk of her developing anxiety during her pregnancy. Further it can impart various benefits to her child: increased cognition, increased receptive language skills, fine motor skills, and gross motor skills. Also lycopene administered to the pregnant woman can increase expressive language in the child. Pre-natal supplements are also included in this invention.

### CROSS REFERENCE TO RELATED APPLICATIONS

This application claims the benefit of the filing date of Singapore National Patent Application No. 10201900604T filed 23 Jan. 2019, the disclosure of which is hereby incorporated herein by reference.

### BRIEF SUMMARY OF THE INVENTION

This invention relates to the administration of beta-cryptoxanthin to an expectant mother, for the beneficial effects to her child. For example, beta-cryptoxanthin increased cognition scores, as well as fine motor skills and gross motor skills in the children. This invention also relates to the prenatal use of lycopene, which increased expressive language skills in the child.

### BACKGROUND OF THE INVENTION

Beta-cryptoxanthin is a known carotenoid, present in mother's milk, and in some fruits and vegetables, but is not often used in nutritional supplements.

WO05/110122 (DSM IP Assets, B.V.) discloses the use of beta-cryptoxanthin for use in promoting protein synthesis in an individual. Optionally, other carotenoids may be co-administered for this purpose.

Also there has been some investigation involving stimulation of bone formation with B-cryptoxanthin.

There have been reports of the association between antioxidants in general, and cognition enhancement. However, none have looked at the influence beta-cryptoxanthin has on childhood cognition. It would be desirable to provide supplements to an expectant mother which would enhance her child's cognition and other developmental skills.

### DETAILED DESCRIPTION OF THE INVENTION

We have found, according to this invention that there is a correlation between the levels of beta-cryptoxanthin in an expectant mother's blood plasma and a number of indices of cognition and developmental indices of her child. Higher maternal β-cryptoxanthin was positively and statistically significantly associated with higher cognitive scores, higher receptive language scores, higher fine motor scores, and higher gross motor scores of the offspring measured 24 months after birth. In addition, it was found that lycopene increases the expressive language skills of the child.

Thus, one aspect of this invention is a method of improving the cognition, receptive language ability, fine motor skills and/or gross motor skills of a child comprising administering to its expectant mother an effective amount of beta-cryptoxanthin during the mother's pregnancy. Another aspect is pre-natal beta-cryptoxanthin for non-therapeutically improving the cognition, receptive language ability, fine motor skills and/or gross motor skills of a healthy child. Another aspect is beta-cryptoxanthin for the manufacture a pre-natal medicament, or nutraceutical for improving the cognition, receptive language ability, fine motor skills and/or gross motor skills of a healthy child.

Another aspect of this invention is a pre-natal composition comprising an effective amount of beta-cryptoxanthin. In some embodiments, the beta-cryptoxanthin is combined with at least one further carotenoid selected from the group consisting of: beta carotene, lutein, zeaxanthin, and lycopene. Another embodiment of this invention is a pre-natal supplement which comprises beta cryptoxanthin and at least one further supplement which is administered during pregnancy, selected from the group consisting of iron, folic acid, B-Vitamins such as Vitamin B6, and B12, calcium, and Vitamin D.

### Definitions

As used throughout the specification and claims, the following definitions apply:

“The Bayley Scales” means The Bayley Scales of Infant and Toddler Development, 3rd edition (BSID-III) 2006 is a widely used comprehensive validated assessment of infant skills. It has various “scales” which assess aspects of development, including:
Cognitive Scale, which assesses play skills; information processing (attention to novelty, habituation, memory), and problem-solving
Language Scale, which contains receptive and expressive language subtests to assess communication skills including language and gestures
Motor Scale, which assesses fine motor development and gross motor development.

“Improved” means that the score a child reaches on the Bayley Scales reflects more highly developed skills as compared to the scores of children whose mothers had lower beta-cryptoxanthin concentrations in her plasma.

“β-cryptoxanthin” includes β-cryptoxanthin either from natural source or synthetically prepared R cryptoxanthin. β-Cryptoxanthin (more specifically, (all-E) β-cryptoxanthin) from natural source may contain β-cryptoxanthin esters with saturated and unsaturated fatty acids, (mainly laurate, myristate, palmitate, stearate, linolate) as well as the isomers (preferably 7′, 9′,11′ and 13′ β-cryptoxanthin) which are included also for use in the present invention. In a preferred aspect, synthetically prepared (all-E)-β-cryptoxanthin is used for the purposes of the invention.

The Bayley Scales of Infant and Toddler Development, 3rd edition (BSID-III) 2006 was administered to infants at 24 (±1) months, in their homes when infants were likely to be alert. This is a standardized test that assesses development of children 1-42 months of age in the following domains: cognitive, receptive and expressive language, and fine and gross motor skills. The test was administered in English, Chinese, Malay or Tamil languages depending on the child's dominant language.

We found, in accordance with this invention that women with lower concentrations of β-cryptoxanthin tended to have probable anxiety. Thus, one embodiment of this invention is a method of improving the mood of a pregnant woman experiencing anxiety related symptoms, or lessening the risk of a pregnant woman experiencing anxiety comprising administering to the woman an effective amount of beta-cryptoxanthin. Another embodiment of this invention is the non-therapeutic use of beta-cryptoxanthin in pregnant women to improve mood related to feeling anxious. Another embodiment of this invention is the use of beta cryptoxanthin in the manufacture of a nutraceutical or pharmaceutical medicament to lessen the risk of a pregnant woman experiencing anxiety.

Thus, another embodiment of this invention is a method of improving development in a child, comprising antenatally administering an effective amount of beta-cryptoxanthin to the child's mother, wherein the improvement is in cognition. Another embodiment of this invention is the non-therapeutic use of prenatal beta-cryptoxanthin for the improved cognition in a child. Another aspect of this invention is the use of beta-cryptoxanthin in the manufacture of a pre-natal nutritional supplement or medicament for the improved cognition in a child.

The prenatal administration to the mother may support the child's ability to think, learn and memorize. In addition, it may support the cognitive development of the child.

Thus, another embodiment of this invention is a method of improving development in a child, comprising antenatally administering an effective amount of beta-cryptoxanthin to the child's mother, wherein the improvement is in receptive language. Receptive language is the ability to understand language, and includes the ability to understand words, sentences and the meaning of what others say or what is read. Another embodiment of this invention is the non-therapeutic use of prenatal beta-cryptoxanthin for the improving receptive language skills in a child. Another aspect of this invention is the use of beta-cryptoxanthin in the manufacture of a pre-natal nutritional supplement or medicament for improving receptive language in a child.

Thus, another embodiment of this invention is a method of improving development in a child, comprising antenatally administering an effective amount of lycopene to the child's mother, wherein the improvement is in expressive language. Another embodiment of this invention is the non-therapeutic use of prenatal lycopene for the improving expressive language skills in a child. Another aspect of this invention is the use of lycopene in the manufacture of a pre-natal nutritional supplement or medicament for improving expressive language in a child. The lycopene may be administered together with beta-cryptoxanthin, or it may be administered on its own. Preferably it is administered along with beta-cryptoxanthin so that additional benefits to the mother and child are also occurring.

Prenatal administration of beta-cryptoxanthin and/or lycopene may support listening and comprehension skills of a child, and may support expressive communication of the child.

Thus, another embodiment of this invention is a method of improving development in a child, comprising antenatally administering an effective amount of beta-cryptoxanthin to the child's mother, wherein the improvement is in expressive language. Expressive language includes the ability to put thoughts into words and sentences in a way which makes sense and is grammatically accurate. Another embodiment of this invention is the non-therapeutic use of prenatal beta-cryptoxanthin for non-therapeutically improving expressive language skills in a child. Another aspect of this invention is the use of beta-cryptoxanthin in the manufacture of a pre-natal nutritional supplement or medicament for the improving expressing language in a child.

Thus, another embodiment of this invention is a method of improving development in a child, comprising antenatally administering an effective amount of beta-cryptoxanthin to the child's mother, wherein the improvement is in the child's fine motor skills. Fine motor skills are involved in coordination of small movements that occur in wrists, hands, fingers, feet and toes. Another embodiment of this invention is the non-therapeutic use of prenatal beta-cryptoxanthin for the improved fine motor skills in a child. Another aspect of this invention is the use of beta-cryptoxanthin in the manufacture of a pre-natal nutritional supplement or medicament for the improved fine motor skills in a child.

The prenatal administration of beta cryptoxanthin may support the development of coordination and fine muscle movements of the child.

Thus, another embodiment of this invention is a method of improving development in a child, comprising antenatally administering an effective amount of beta-cryptoxanthin to the child's mother, wherein the improvement is in gross motor skills. Gross motor skills are involved in the movement and coordination of the arms, legs, and other large body parts. This would include actions such as running, crawling, and swimming. Another embodiment of this invention is the non-therapeutic use of prenatal beta-cryptoxanthin for the improved gross motor skills in a child. Another aspect of this invention is the use of beta-cryptoxanthin in the manufacture of a pre-natal nutritional supplement or medicament for the improved gross motor skill in a child.

Prenatal administration of beta-cryptoxanthin may support the development of gross motor skills of the child, and may support the development of gross motor skills such as balance, coordination, physical strength, body awareness and reaction time of the child.

### Dosages

In accordance with the present invention, β-cryptoxanthin is suitably administered in dosages up to about 50 mg/day, more particularly, from about 100 μg/day to about 30 mg/day, especially from about 1 mg/day to about 10 mg/day for a human adult of about 70 kg body weight. In a preferred embodiment, the beta-cryptoxanthin is administered daily from at least the second trimester of pregnancy to the time of delivery; in a more preferred embodiment, the administration begins during the first trimester; and even more preferably the administration begins prior to the pregnancy occurring. In preferred embodiments, the administration lasts throughout lactation.

For lycopene, the dosages are the same as those outlined for beta-cryptoxanthin above.

The beta-cryptoxanthin and lycopene may be taken in a single dosage form or may be administered separately. Another embodiment of this invention is a composition comprising beta-cryptoxanthin and lycopene as the sole active ingredients.

For the purposes of the invention, β-cryptoxanthin and/or lycopene is suitably provided in compositions for oral administration which may be solid or liquid galenical formulations, dietary compositions, pharmaceuticals, or food. Examples of solid galenical formulations are tablets, capsules (e.g. hard or soft shell gelatin capsules), pills, sachets, powders, granules and the like which contain the active ingredient together with conventional galenical carriers. Any conventional carrier material can be used. The carrier material can be organic or inorganic inert carrier material suitable for oral administration. Suitable carriers include water, gelatin, gum arabic, lactose, starch, magnesium stearate, talc, vegetable oils, and the like. Additionally additives such as flavouring agents, preservatives, stabilizers, emulsifying agents, buffers and the like may be added in accordance with accepted practices of pharmaceutical compounding. Additional active ingredients for co-administration with β-cryptoxanthin and/or lycopene may administered, together with β-cryptoxanthin and/or lycopene in a single composition, or may be administered in individual dosage units. Dietary compositions comprising β-cryptoxanthin and/or lycopene can be beverages, instant beverages, or food supplements.

The following non-limiting Examples are presented to better illustrate the invention.

### EXAMPLES

### Methods

### Subjects

Data for the present analysis were from the Growing Up in Singapore Towards healthy Outcomes (GUSTO) study a prospective mother-offspring cohort in Singapore (34). Detailed descriptions of the GUSTO study have been published Soh, et al 2014 Int. J Epidemiology 43(5):1401-9, which is hereby incorporated by reference. In brief, pregnant women (≥18 years) in their first trimester (<14 weeks) were recruited from the National University Hospital (NUH) and KK Women's and Children's Hospital (KKH) between June 2009 and September 2010. To be eligible for the study, the pregnant woman had to: 1) be a Singapore citizen or permanent residents of Chinese, Malay or Indian ethnicity with homogenous parental ethnic background; 2) have an intention to deliver in the two hospitals and to reside in Singapore for the next five years; and 3) consent to donate birth tissues at delivery. Women receiving chemotherapy, psychotropic drugs or were diagnosed with type-1 diabetes were not eligible to participate. The GUSTO study has received ethical approval from the Institutional Review Board of NUH and KKH, and all procedures were conducted according to the guidelines laid down in the Declaration of Helsinki. Written informed consent was obtained from all participants at study recruitment.

A total of 1247 pregnant women participated at baseline. The present analysis included pregnant women who had singleton live births (n=1237), in whom plasma carotenoids concentrations were analyzed (n=701), and their offspring completed neurocognitive assessments at 24 months (n=361).

Maternal plasma carotenoids concentrations Non-fasting bloods samples were obtained from mothers during delivery using venipuncture technique. The blood samples were processed within 4 hours, stored at −80° C. and thawed prior to analysis. Ultra High Performance Liquid Chromatography (UPLC) method was used to determine plasma concentrations of individual carotenoids (α-carotene, β-carotene, β-cryptoxanthin, lutein, lycopene, and zeaxanthin), and quantified using Photo-Diode Array detection. The precision of the method was examined using pooled and spiked plasma samples and the results were similar as published earlier with the RSDs (n=6) of within day assays and between-day assays are generally <10% and <15%, respectively (Lee et al 2009 J. Chromatog. A 1216(15):3131-7).

An Ultra High Performance Liquid Chromatography (UPLC) method was used to determine plasma concentrations of retinol and individual carotenoids (α-carotene, β-carotene, β-cryptoxanthin, lutein, lycopene, and zeaxanthin). The UPLC is a special variant of a previously established HPLC method (Lee B L, et al J Chromatogr A. 2009; 1216(15):3131-7), but using column with particle sizes less than 2.6 μm to allow for better separation and faster analysis.

The UPLC method is as follows: in an amber micro-centrifuge tube, a 30 μL aliquot of plasma was deprotienized with equal volume of EB solution (ethanol-tert-butanol, 4:1, v/v) and I.S. (echinenone, 0.4 mg/L). It was then extracted with 100 μL of n-hexane for 2 min. After centrifugation (15 000 g/1 min), 160 μL of supernatant was transferred into another amber micro-centrifuge tube and dried under a stream of nitrogen. The dried residue was reconstituted in 60 μL of EB solution and 5 μL was injected onto a Kinetex C18 core-shell (2.6 μm, 100 mm×4.6 mm ID; Phenomenex). The four mobile phase solutions used for gradient separation were: A, pure acetonitrile; B, pure methanol; and C, a mixture of ethanol and tert-butanol (8:2, v/v) and D, pure water. Using a Waters Acquity H-class UPLC system, the gradient separation was initiated with 100% D at a constant flow rate of 0.6 ml/min and linearly changed to 100% B within 0.1 min, 10% A and 90% B from 0.1-6 min, 40% A and 60% C from 6-8 min and 100% C from 10-14 min. The column was then re-equilibrated with water (100% D) for 5 min before the next injection of sample.

### Cognitive Outcomes in Infants

The Bayley Scales of Infant and Toddler Development, 3rd edition (BSID-III) 2006 was administered to infants at 24 (±1) months, in their homes when infants were likely to be alert. This is a standardized test that assesses development of children 1-42 months of age in the following domains: cognitive, receptive and expressive language, and fine and gross motor skills. The test was administered in English, Chinese, Malay or Tamil languages depending on the child's dominant language.

Administration and scoring was performed by research coordinators trained by the head psychologist from KKH in accordance to the manual. Raw test scores were used as age-specific norms were not available for our population.

### Covariates

Information on maternal age and self-reported ethnicity and highest education attained were collected during recruitment visit (<14 weeks' gestation). At the 26-28 weeks' gestation clinic visit, antenatal mental well-being were assessed with the Edinburgh Postnatal Depression Scale (EPDS) (Cox et al 1987 The British J. Psychiatry: J Mental Sci 150:782-6) and the State-Trait Anxiety Inventory (STAI) (Spielberger. State-trait Anxiety Inventory: A comprehensive Bibliography: Consulting Psychologists Press, 1984). Maternal pre-pregnancy BMI was based on self-reported pre-pregnancy weight, and height measured with a stadiometer (SECA model 213) at the 26-28 weeks' gestation clinic visit, calculated as weight divided by height squared (kg/m²). Maternal parity, infant gestational age (determined by a dating ultrasound scan in the first trimester) and birth weight (measured by midwives within 72 hours of delivery) were retrieved from hospital delivery records. Mothers' breastfeeding practices were obtained during postnatal visits by trained interviewers and duration of any breastfeeding was categorized as follows: no breastfeeding, <1 month, 1 to ≤3 months, 3 to <6 months, 6 to <12 months and ≥1.2 months.

### Statistical Analysis

Maternal plasma concentrations of individual carotenoids were summarized according to maternal and infant characteristics for the 419 mother-offspring pairs with data for the cognitive test. Differences in concentrations between groups were compared using non-parametric analyses Wilcoxon rank-sum and Kruskal-Wallis tests, as data were not normally distributed. Bonferroni post hoc analysis was performed to identify groups which differed if the Kruskal Wallis test was significant. The values for individual maternal plasma carotenoids were log-transformed, then converted to standard deviation scores (SDS) for easier interpretation. The BSID-III raw scores were also converted to SDS to facilitate comparison across the cognitive tests and domain scales.

Associations of each maternal plasma carotenoids with each BSID-III scale in the children were examined using linear regressions. Several statistical models were employed: Model 1 basic model with adjustment for infant's exact age at cognitive testing; Model 2 additional adjustment for maternal age, ethnicity, education, pre-pregnancy BMI, antenatal depression and anxiety levels (potential confounders). We considered Model 2 to be the main model. Further adjustment for infant gestational age, birth weight and breastfeeding duration was performed to examine if any associations found were acting through these factors (Model 3).

Missing data for covariates were imputed using multiple imputation technique with chained equations (20 times) for the following confounding variables: n=3 maternal education, n=7 EPDS, n=21 STAI, n=40 maternal pre-pregnancy BMI, n=5 infant birthweight and n=10 breastfeeding duration. All analyses were performed using Stata version 14 (StataCorp LP, College Station, Tex., USA). To account for multiple testing, P<0.01 was considered statistically significant.

### Results

### Characteristics of Mother-Offspring Pairs

Pregnant women of Malay ethnicity tended to have lower concentrations of α-carotene and lutein, while women of Indian ethnicity were more likely to have lower concentrations of β-carotene, β-cryptoxanthin, zeaxanthin and lycopene. Those with lower concentrations of α-, β-carotene and lutein tended to attain lower educational level, more likely to be obese and to have probable depression and anxiety, and also tended to breastfeed for a shorter duration. Additionally, women with lower concentrations of α-carotene tended to be younger; women with lower concentrations of β-cryptoxanthin tended to have probable anxiety; and those with lower concentrations of zeaxanthin tended to be older and attained higher education level. On the other hand, pregnant women with lower concentrations of lycopene tended to have probable depression, to be primiparous, and to have breastfed for 1-3 months.

### Maternal Plasma Carotenoids and BSID-III Outcomes in Infant at 24 Months

The associations of maternal plasma carotenoids concentrations with scores of each of the BSID-III scales in infants at 24 months are presented in Table 1.

TABLE 1
Associations of maternal plasma carotenoids concentrations with scores of Bayley Scale of Infant and Toddler
Development -III at 24 months of age in the Growing Up in Singapore Towards healthy Outcomes study (n = 361).

| | | | | Expressive | | | | | |
|---|---|---|---|---|---|---|---|---|---|
| Cognitive | | Receptive language | | Language | | Fine motor | | Gross motor | |
| β (95% CI) | P | β (95% CI) | P | β (95% CI) | P | β (95% CI) | P | β (95% CI) | P |

α-carotene

| | | | | | | | | | | |
|---|---|---|---|---|---|---|---|---|---|---|
| Model 1 | 0.06 (−0.03, | 0.196 | 0.09 (−0.01, | 0.075 | 0.10 (0.01, | 0.038 | 0.08 (−0.01, | 0.086 | 0.08 (−0.02, | 0.105 |
| | 0.16) | | 0.19) | | 0.20) | | 0.18) | | 0.18) | |
| Model 2 | −0.01 (−0.11, | 0.900 | 0.002 (−0.10, | 0.970 | 0.06 (−0.05, | 0.288 | 0.06 (−0.04, | 0.245 | 0.06 (−0.04, | 0.250 |
| | 0.10) | | 0.11) | | 0.16) | | 0.17) | | 0.17) | |
| Model 3 | −0.04 (−0.14, | 0.516 | −0.04 (−0.14, | 0.454 | 0.02 (−0.08, | 0.654 | 0.05 (−0.06, | 0.343 | 0.04 (−0.07, | 0.525 |
| | 0.07) | | 0.06) | | 0.13) | | 0.16) | | 0.14) | |

β-carotene

| | | | | | | | | | | |
|---|---|---|---|---|---|---|---|---|---|---|
| Model 1 | 0.22 (0.11, | <0.001 | 0.23 (0.12, | <0.001 | 0.18 (0.08, | 0.001 | 0.14 (0.04, | 0.008 | 0.15 (0.04, | 0.006 |
| | 0.32) | | 0.33) | | 0.29) | | 0.25) | | 0.26) | |
| Model 2 | 0.16 (0.04, | 0.007 | 0.15 (0.04, | 0.010 | 0.14 (0.03, | 0.017 | 0.12 (0.00005, | 0.050 | 0.14 (0.02, | 0.019 |
| | 0.28) | | 0.26) | | 0.26) | | 0.24) | | 0.26) | |
| Model 3 | 0.13 (0.01, | 0.029 | 0.11 (−0.01, | 0.070 | 0.10 (−0.02, | 0.088 | 0.10 (−0.02, | 0.096 | 0.12 (−0.003, | 0.057 |
| | 0.25) | | 0.22) | | 0.22) | | 0.22) | | 0.24) | |

β-cryptoxanthin

| | | | | | | | | | | |
|---|---|---|---|---|---|---|---|---|---|---|
| Model 1 | 0.21 (0.11, | <0.001 | 0.21 (0.10, | <0.001 | 0.15 (0.05, | 0.004 | 0.17 (0.07, | 0.001 | 0.17 (0.06, | 0.001 |
| | 0.31) | | 0.31) | | 0.25) | | 0.27) | | 0.27) | |
| Model 2 | 0.18 (0.08, | <0.001 | 0.17 (0.07, | 0.001 | 0.13 (0.03, | 0.012 | 0.16 (0.06, | 0.003 | 0.16 (0.06, | 0.002 |
| | 0.28) | | 0.27) | | 0.23) | | 0.26) | | 0.27) | |
| Model 3 | 0.18 (0.08, | 0.001 | 0.16 (0.06, | 0.001 | 0.12 (0.02, | 0.019 | 0.15 (0.05, | 0.004 | 0.15 (0.05, | 0.005 |
| | 0.28) | | 0.26) | | 0.22) | | 0.26) | | 0.25) | |

Lutein

| | | | | | | | | | | |
|---|---|---|---|---|---|---|---|---|---|---|
| Model 1 | 0.03 (−0.08, | 0.555 | 0.08 (−0.03, | 0.140 | 0.004 (−0.11, | 0.938 | 0.01 (−0.10, | 0.834 | 0.03 (−0.08, | 0.609 |
| | 0.14) | | 0.19) | | 0.11) | | 0.12) | | 0.14) | |
| Model 2 | −0.08 (−0.21, | 0.223 | −0.04 (−0.17, | 0.484 | −0.09 (−0.21, | 0.187 | 0.02 (−0.15, | 0.735 | −0.02 (−0.15, | 0.822 |
| | 0.05) | | 0.08) | | 0.04) | | 0.11) | | 0.12) | |
| Model 3 | −0.11 (−0.24, | 0.098 | −0.08 (−0.21, | 0.184 | −0.12 (−0.25, | 0.054 | −0.04 (−0.17, | 0.542 | −0.04 (−0.17, | 0.585 |
| | 0.02) | | 0.04) | | 0.002) | | 0.09) | | 0.09) | |

Zeaxanthin

| | | | | | | | | | | |
|---|---|---|---|---|---|---|---|---|---|---|
| Model 1 | 0.01 (−0.10, | 0.894 | −0.10 (−0.21, | 0.062 | −0.09 (−0.20, | 0.093 | 0.04 (−0.07, | 0.508 | 0.02 (−0.09, | 0.764 |
| | 0.11) | | 0.005) | | 0.02) | | 0.14) | | 0.12) | |
| Model 2 | 0.05 (−0.06, | 0.360 | −0.05 (−0.15, | 0.361 | −0.05 (−0.16, | 0.336 | 0.05 (−0.06, | 0.362 | 0.04 (−0.07, | 0.449 |
| | 0.16) | | 0.06) | | 0.05) | | 0.16) | | 0.15) | |
| Model 3 | 0.05 (−0.05, | 0.316 | −0.04 (−0.15, | 0.414 | −0.04 (−0.15, | 0.404 | 0.05 (−0.05, | 0.325 | 0.05(−0.06, | 0.354 |
| | 0.16) | | 0.06) | | 0.06) | | 0.16) | | 0.16) | |

Lycopene

| | | | | | | | | | | |
|---|---|---|---|---|---|---|---|---|---|---|
| Model 1 | 0.14 (0.03, | 0.009 | 0.11 (0.003, | 0.044 | 0.15 (0.05, | 0.004 | 0.04 (−0.07, | 0.488 | 0.09 (−0.02, | 0.105 |
| | 0.24) | | 0.21) | | 0.25) | | 0.14) | | 0.19) | |
| Model 2 | 0.12 (0.02, | 0.017 | 0.09 (−0.01, | 0.085 | 0.15 (0.05, | 0.004 | 0.03 (−0.08, | 0.620 | 0.08 (−0.02, | 0.119 |
| | 0.23) | | 0.19) | | 0.25) | | 0.13) | | 0.19) | |
| Model 3 | 0.11 (0.01, | 0.030 | 0.07 (−0.03, | 0.155 | 0.14 (0.04, | 0.008 | 0.02 (−0.08, | 0.688 | 0.09 (−0.01, | 0.083 |
| | 0.22) | | 0.17) | | 0.24) | | 0.12) | | 0.20) | |

Effect estimates are per SD increment in log-transformed maternal plasma carotenoids concentrations, and per SD BSID-III score
Model 1 - adjusted for infant's age at cognitive testing
Model 2 - adjusted as for Model 1 and maternal age, ethnicity, education, pre-pregnancy BMI, parity, antenatal depression and anxiety levels.
Model 3 - adjusted as for Model 2 and infant gestational age, birth weight and breastfeeding duration

Higher maternal β-carotene concentrations (per SD increment in log-transformed concentrations or 0.841 mg/L) was associated with 0.16 SD (95% CI: 0.04, 0.28) higher cognitive scores after adjusting for key confounders (Model 2). This association was attenuated after adjusting for breastfeeding duration, infant gestational age and birth weight (Model 3), in which breastfeeding duration was observed to be the main attenuating factor in stepwise adjustment. In contrast, significant associations between higher maternal β-carotene concentrations and higher scores in receptive and expressive language, and fine and gross motor were attenuated after adjusting for confounders.

Additionally, after adjustment for confounders, higher maternal β-cryptoxanthin (per SD increment in log-transformed concentrations or 0.675 mg/L) was associated with 0.18 SD (95% CI: 0.08, 0.28) higher cognitive scores, 0.17 SD (95% CI: 0.07, 0.27) higher receptive language scores, 0.16 SD (95% CI: 0.06, 0.26) higher fine motor scores, and 0.16 SD (95% CI: 0.06, 0.27) higher gross motor scores (Model 2). These associations remained statistically significant after adjustment for potential mediators (Model 3). The association between maternal β-cryptoxanthin and expressive language scores, however, were attenuated after adjusting for confounders.

We observed higher maternal lycopene (per SD increment in log-transformed concentrations or 0.522 mg/L) to be associated with 0.15 SD (95% CI: 0.05, 0.25) higher expressive language scores after adjustment for confounders, and the association remained significant after adjusting for potential mediators. The association between maternal lycopene and cognitive scores was attenuated after adjusting for confounders.

### SUMMARY

Higher maternal β-cryptoxanthin concentrations were most consistently associated with better cognitive development in infants at 24 months, as we observed significant associations with all BSID-III scales after adjustment for confounders (trending significance with expressive language).

### 1. Lycopene Findings

Higher maternal lycopene concentrations were associated with better performance in expressive language at 24 months, but not with receptive language

### 2. Strengths

First study to examine associations between maternal carotenoids and cognitive development in children
We showed that specific carotenoids rather than vitamin A or retinol play important roles in early neurocognitive development; specifically potential role of β-cryptoxanthin in neurodevelopment which has not been well-studied.

1. A method of improving the cognition, receptive language ability, expressive language ability, fine motor skills and/or gross motor skills of a child comprising administering to its expectant mother an effective amount of beta-cryptoxanthin during her pregnancy.

2. The method of claim 1 where the improvement is measurable at 24 months.

3. A prenatal composition comprising an effective amount of beta cryptoxanthin.

4. a method of improving the mood of a pregnant woman experiencing anxiety related symptoms, or lessening the risk of a pregnant woman experiencing anxiety comprising administering to the woman an effective amount of beta-cryptoxanthin.

5. A method of improving the expressive language ability of a child comprising administering to its expectant mother an effective amount of lycopene.
